(19) United States
(12) Patent Application Publication
(10) Pub. No.: US 2013/0324608 A1
(43) Pub. Date: Dec. 5, 2013

(54) METHODS AND COMPOSITIONS FOR TREATING, REDUCING OR PREVENTING DAMAGE TO THE NERVOUS SYSTEM OF ANIMALS

(71) Applicant: Yuanlong Pan, Chesterfield, MO (US)

(72) Inventor: Yuanlong Pan, Chesterfield, MO (US)

(21) Appl. No.: 13/985,884

(22) PCT Filed: Feb. 7, 2012
(86) PCT No.: PCT/US12/24137

§ 371 Date: Aug. 16, 2013

Related U.S. Application Data
(60) Provisional application No. 61/463,594, filed on Feb. 18, 2011.

(51) Int. Cl. A61K 31/202; A61K 45/06; A23L 1/30; A61K 31/198

(52) U.S. Cl. CPC A61K 31/202; A61K 31/198; A61K 45/06; A23L 1/3008
USPC 514/560

(57) ABSTRACT

Methods and compositions for treating, reducing, or preventing damage to at least one component of the nervous system of an animal are disclosed. The methods comprise administering to the animal a composition comprising UFA and NORC in an amount effective to treat, reduce, or prevent damage to at least one component of the nervous system. Methods extending the prime years of an animal's life, improving the quality of life, and promoting health and wellness of an animal using compositions comprising UFA and NORC, and optionally, antioxidant(s) and/or B vitamins are also disclosed.

### CROSS REFERENCE TO RELATED APPLICATIONS

This application claims priority to U.S. Provisional Application Ser. No. 61/463,594 filed Feb. 18, 2011, the disclosure of which is incorporated herein by this reference.

### BACKGROUND OF THE INVENTION

1. Field of the Invention

The invention relates generally to methods and compositions for treating, reducing, or preventing damage to the nervous system of animals and particularly to methods and compositions using unsaturated fatty acids and nitric oxide releasing compounds for treating, reducing, or preventing damage to the nervous system of animals.

2. Description of Related Art

The nervous system is a complex network of cells, tissues, and organs that regulate the body's responses to internal and external stimuli. In vertebrates, it consists of the brain, spinal cord, nerves, ganglia, and parts of the receptor and effector organs. In mammals, the nervous system consists of the central nervous system and the peripheral nervous system. The central nervous system consists of the brain and spinal cord, both of which are surrounded by protective connective tissues. The peripheral nervous system consists of nerves that carry impulses to and from the central nervous system, and includes both sensory (afferent) pathways that receive signals from the body to the central nervous system, as well as motor (efferent) pathways that relay signals from the central nervous system to the body. These pathways are controlled by somatic and autonomic nervous systems. The somatic nervous system comprises all nerves controlling the muscular system and external sensory receptors. The autonomic nervous system consists of motor neurons that control internal organs, is involved in maintaining homeostasis, and is itself divided into two parts, the sympathetic and the parasympathetic systems. The sympathetic nervous system is primarily involved with “fight or flight” and other responses to stressors, while the parasympathetic nervous system controls the ‘relaxation’ response and will, e.g., tend to restore homeostasis after a stress response.

The properly functioning nervous system allows the animal to live, breathe, interact with its environment, reproduce, etc. However, the nervous system is susceptible to damage from a variety of sources including physical injury, disease, and other damage. Either or both of the central or peripheral nervous systems can be injured or damaged and there are varying degrees of such damage.

Physical injury can arise from nervous system injury caused by trauma, e.g., injuries from daily or routine activities, sports or competitive injuries, burns, electrical shock, cold-exposure, or damage due to overuse, repetitive use, or nonuse. Another common source of injury is accident trauma; such as automobile or industrial accidents which often result in nervous system injury, lacerations, gunshots, and the like. As an example of physical injury in humans, blows to the head received by competitors in combat sports (e.g., boxing), football players, hockey players, especially where severe, repeated over time, or left untreated, may result in nervous system injury, particularly to the brain or spinal cord. Improvements in safety gear, diagnosis, and treatment, as well as changes in rules to protect the athletes are ostensibly designed to mitigate the risk injuries. Compressive injuries are well known, including the repetitive use type (e.g., carpal tunnel syndrome), and crushing accidents.

Disease is another common source of injury to the nervous system. A variety of diseases can cause such injury or insult to the central or peripheral nervous systems, as either a primary or secondary result. Many infectious and/or inflammatory conditions can result in damage to the nervous system, such as various types of neuropathies. Examples of such infectious or inflammatory diseases include Lyme disease, diphtheria, HIV/AIDS, leprosy, shingles (i.e., herpes varicella zoster) and other Herpes infections, hepatitis B, hepatitis C, and certain neoplastic diseases are all known to cause neuropathy or other damage to the nervous system. Various other diseases, such as certain autoimmune conditions and/or possible infections for which no causative agent is known can also lead to neuropathic problems. Examples of such diseases include sarcoidosis, Guillain-Barré Syndrome, Acute Inflammatory Demyelinating Polyneuropathy (AIDP), Chronic Inflammatory Demyelinating Polyneuropathy (CIDP), Celiac disease, and multifocal motor neuropathy. Related vascular conditions such as polyarteritis nodosa (PAN), rheumatoid arthritis, systemic lupus erythematosus (Lupus), and Sjögren's Syndrome, have been associated with neuropathic injury. Yet other diseases that can produce neuropathic injury include peripheral neuropathies associated with protein abnormalities such as monoclonal gammopathy, amyloidosis, cryoglobulinemia, and POEMS.

A third category of injury to the nervous system, is perhaps more general and includes both systemic and metabolic conditions. Systemic conditions include, for example, intoxication (i.e., exposure to toxins), chemical exposure (such as accidental exposure, drug use and/or abuse, etc), alcohol use, and the like. Relevant metabolic conditions include diabetes mellitus, hypoglycemia, uremia, hypothyroidism, hepatic failure, polycythemia, amyloidosis, acromegaly, porphyria, disorders of lipid/glycolipid metabolism, metabolic syndrome, hormone imbalances (thyroid hormone, growth hormone), nutritional/vitamin deficiencies (vitamins B-1, B-6, B-12, niacin, thiamine, E), and mitochondrial disorders. These conditions can all involve the peripheral nerves by altering the structure or function of myelin and axons due to metabolic pathway dysregulation. In addition, there is a certain amount of age-related decline in various nervous system functions, particular certain sensory functions such as hearing (loss, tinnitus), taste, smell, vision, tactile senses (e.g., loss of fine touch, vibration, pain insensitivity, pain hypersensitivity, numbness), balance, motor control, and others. In addition, the presence of various physiological changes can be seen as a function of aging including tangles, plaques, atrophy, pigment (lipofuscin) deposits, and the like; in some cases these are associated with nerve system damage, such as certain types of dementia. A number of neuropathic conditions have no known cause or apparent underlying etiology and as such are deemed “idiopathic” neuropathies. Such conditions can be particularly frustrating for the inflicted animal, as well as for doctors, health care providers and the like as it may be difficult to develop a treatment plan.

As can be seen, there are many types and sources of injury or damage to the nervous system and/or nerves. For example, statistics from the U.S. National Institute of Neurological Disorders and Stroke reveal over 100 types of peripheral neuropathies. Such damage may be acute or chronic.

While there is no single classification system that defines all possible types of nerve injury, such systems generally correlate the extent of injury with symptoms, pathology, and likely prognosis. Two well known systems are Seddon's and Sunderland's classifications for peripheral nerve injuries. In each, peripheral nerve injuries are classified based on three main types of nerve fiber injury, whether there is continuity of the nerve, and likelihood of recovery. In Seddon's classic system, there are three types of nerve injury, which are, in order of increasing severity, neurapraxia, axonotmesis, and neurotmesis. Sunderland grades injuries on a scale of I-V, with grades I and II identical to the first two classes of Seddon. Sunderland's grades III, IV, and IV are all encompassed within Seddon's neurotmesis.

Neurapraxia is the least severe form of nerve injury because the physical structure of the nerve remains intact. Most commonly, this involves compression of the nerve or disruption to the blood supply, e.g., ischemia, resulting in an interruption in conduction of the impulse down the nerve fiber. Generally, there is only a temporary loss of function, which is reversible. Because the nerve damage is not extensive, degeneration of the myelin sheath layer (i.e., “Wallerian degeneration”) generally does not occur. Such degeneration is a prelude to nerve regeneration or rebuilding when needed. Thus, recovery from neurapraxia does not require regeneration of the damaged nerves. Neurapraxia frequently features greater involvement of motor function rather than sensory function, and autonomic function is also retained. Complete recovery from this type of injury is expected and no surgical intervention is indicated.

Axonotmesis is a more severe nerve injury with disruption of the neuronal axon, but maintenance of the myelin sheath. This type of nerve damage may cause paralysis of the motor, sensory, and autonomic nervous systems. This type of injury is typically caused by force, e.g., from a crash, blow, or similar incident. If the force creating the nerve damage is removed, the axon may regenerate and completely recover. Axonotmesis involves loss of the relative continuity of the axon and its covering of myelin. Because axonal continuity is lost, Wallerian degeneration occurs. Loss in both motor and sensory spines is more complete with axonotmesis than with neurapraxia. Recovery occurs through regeneration of the axons. Axonotmesis is usually the result of a more severe crush or contusion than neurapraxia but can also occur when the nerve is stretched. There is usually an element of retrograde proximal degeneration of the axon. For regeneration to occur, this loss must first be overcome. Regeneration, and thus recovery, usually takes weeks to years, and surgical intervention may be appropriate if the recovery does not continue to progress.

Neurotmesis is the most severe injury with substantial potential for recovery. Generally, neurotmesis results from severe contusion, stretch, or laceration. The axon and the encapsulating connective tissue lose their continuity. Typically, there is a complete loss of motor, sensory, and autonomic function. Wallerian degeneration occurs, and regeneration and recovery require a long time. Recovery is generally variable and not complete.

Traumatic injuries are the most common injuries for central nerve system, i.e., (brain and spinal cord). Traumatic brain injuries (TBI) are injuries caused by an external force. They are classified based on severity (mild, moderate, and severe), anatomical characteristics of the injury (focal or diffuse, extra-axial or intral-axial), and the mechanism of injury (penetrating head injury or nonpenetrating head injury). The American Spinal Injury Association classifies traumatic spinal cord injuries into five categories (A, B, C, D, and E). The damage and cell death caused by traumatic injuries are the consequences of primary and secondary injury. Primary injury and cell death are caused by trauma impact that stretches, compresses, and tears tissue and blood vessels. However, much more damage and cell death are caused by secondary injury. Secondary injury mechanisms include ischemia, hypoxia, edema, free radical damage, and inflammation etc.

Healing or recovery from damage to the nervous system can require degeneration, neuroregeneration, or remyelination. The generation of glia or myelin, and/or neurons or axons, dendrites, synapses or other portions thereof may be involved. In cases of severing, surgical intervention can facilitate healing. The prognosis for healing of damage to the central nervous system is generally dire compared to recovery of peripheral nervous system injury.

Methods for preventing or treating nervous system injury are known in the art. WO2010024432A1 discloses using a TGF-β1 inhibitor such as an anti-TGF-β1 antibody for treating CNS injuries. US20090325920A1 discloses using progesterone for the treatment of a traumatic central nervous system injury. US20080160006A1 discloses in vitro and topical usage of inorganic salts, at least ten essential amino acids, antioxidants, hormones, essential fatty acids, and vitamins for improving neural cell viability in injured nervous systems. US20070203239A1 discloses using potassium-adenosine triphosphate channel blockers for the prevention and treatment of acute injury to the CNS.

On one hand, there are some literature reports regarding the potential for use of essential fatty acids for treatment of neuropathy and also some reports regarding the potential for use of B vitamins for such purpose. On the other hand, there are numerous literature reports implicating nitric oxide, and/or nitric oxide synthase (or its triggers) as causative agent(s) and/or aggravating factor(s) in many types of neuropathy.

While there are known methods intended for use with the nervous system, novel methods for treating, reducing, or preventing damage to the nervous system of animals are needed and would represent an advance in the art.

### SUMMARY OF THE INVENTION

It is, therefore, an object of the invention to provide methods for treating, reducing, or preventing damage to the nervous system of animals, particularly the brain, spinal cord, and peripheral nervous system.

It is another object of the invention to provide methods for treating, reducing, or preventing damage to the nervous system of an animal caused by injury, disease, or aging.

It is a further object of the invention to provide methods for treating, reducing, or preventing damage to the brain of an animal caused by injury, disease, or aging.

It is another object of the invention to provide methods for treating, reducing, or preventing damage to the spinal cord of an animal caused by injury, disease, or aging.

It is another object of the invention to provide methods for treating, reducing, or preventing damage to the peripheral nervous system of an animal caused by injury, disease, or aging.

It is another object of the invention to provide methods for preserving or maintaining the nervous system during the prime years and throughout an animal's life.

It is another object of the invention to provide methods for promoting the health or wellness of animals by treating, reducing, or preventing damage to the nervous system of the animals.

One or more of these or other objects are achieved by identifying an animal susceptible to or suffering from damage to the nervous system and administering in conjunction to the animal one or more unsaturated fatty acids (UFA) and one or more nitric oxide releasing compounds (NORC) in an amount effective for treating, reducing, or preventing damage to the nervous system of the animal. In certain embodiments, one or more B vitamins, one or more antioxidants, or combinations thereof are administered in conjunction with the UFA and NORC in amounts effective for treating, reducing, or preventing damage to at least one component of the nervous system.

Other and further objects, features, and advantages of the invention will be readily apparent to those skilled in the art.

### DETAILED DESCRIPTION OF THE INVENTION

### Definitions

The following abbreviations may be used herein: AA, arachidonic acid; aka, also known as; ALA, alpha-linolenic acid; ANOVA, analysis of variance; CT, computed tomography; DHA, docosahexaenoic acid; DPA, docosapentaenoic acid; E2, estradiol; EMG, electromyography; EPA, eicosapentaenoic acid; FOS, fructooligosaccharide(s); GOS, galactooligosaccharide(s); LA, linoleic acid; L-Arg, L-arginine; MRI, magnetic resonance imaging; NCV, nerve conduction velocity; NO, nitric oxide; NORC, nitric oxide releasing compound(s); OVX, ovariectomized; TENS, Transcutaneous Electrical Nerve Stimulation; TTC, tetrazolium chloride; TUNEL, terminal deoxynucleotidyl transferase dUTP nick end labeling; UFA, unsaturated fatty acid(s), and XOS, xylooligosaccharide(s).

The term “animal” means any animal that can benefit from improvement in, or decrease in the damage to the animal's nervous system, including at least human, avian, bovine, canine, equine, feline, hicrine, lupine, murine, ovine, or porcine animals, and preferably a domesticated animal, and more preferably a companion animal.

The term “companion animals” means domesticated animals such as dogs, cats, birds, rabbits, guinea pigs, ferrets, hamsters, mice, gerbils, pleasure horses, cows, goats, sheep, donkeys, pigs, and more exotic species kept by humans for company, amusement, psychological support, education, physical assistance, extrovert display, and all of the other functions that humans desire or need to share with animals of other species.

The term “individual” when referring to an animal means an individual animal of any species or kind.

The term “unsaturated fatty acid” or “UFA” means one or more polyunsaturated fatty acids and/or monounsaturated fatty acids, including monocarboxylic acids having at least one double bond. UFAs include (n-6) fatty acids such as linoleic acid (LA) and arachidonic acid (AA) and (n-3) fatty acids such as eicosapentaenoic acid (EPA), alpha-linolenic acid (ALA), docosapentaenoic acid (DPA) and docosahexaenoic acid (DHA). UFAs also include myristoleic acid, palmitoleic acid, oleic acid, linoleic acid, cis-vaccenic acid, and erucic acid.

The term “fish oil” means a fatty or oily extract, relatively rich in UFA, whether crude or purified, obtained from a sea animal, preferably a cold-water fish such as, but not limited to, salmon, tuna, mackerel, menhaden, herring, sea bass, striped bass, pollock, flounder, halibut, catfish, lake trout, anchovies, and sardines, as well as shark, swordfish, tilefish, shrimp, and clams, or any combination thereof. “Fish oil” is also a term of art used by ingredient suppliers and encompasses a range of products of varying UFA content and purity.

The term “nitric oxide releasing compounds” or “NORC” means any compound or compounds that cause or can result in the release of nitric oxide in an animal. Examples of such compounds include L-arginine, L-arginine-containing peptides and proteins, and analogs or derivatives thereof that are known or determined to release nitric oxide, such as arginine alpha-ketoglutarate, GEA 3175, sodium nitroprusside, glyceryl trinitrate, S-nitroso-N-acetyl-penicillamine, nitroglycerin, S-NO-glutathione, NO-conjugated non-steroidal anti-inflammatory drugs (e.g., NO-naproxen, NO-aspirin, NO-ibuprofen, NO-Diclofenac, NO-Flurbiprofen, and NO-Ketoprofen), NO-releasing compound-7, NO-releasing compound-5, NO-releasing compound-12, NO-releasing compound-18, diazenium diolates and derivatives thereof, diethylamine NONOate, and any organic or inorganic compound, biomolecule, or analog, homolog, conjugate, or derivative thereof that causes the release of nitric oxide, particularly “free” NO, in an animal. NORC are also defined to include supplements that can be converted to nitric oxide releasing compounds when metabolized in the body, e.g., citrulline and ornithine.

The term “nervous system” includes any or all component(s) that are part of either the central or peripheral nervous system, including the nerves proper (particularly the cranial and spinal nerves), the myelin sheath, and other portions or parts thereof. Preferably, for various embodiments herein, the nervous system means the peripheral nervous system. One of the hallmarks of the peripheral nervous system is the specific source of the myelin coating. As skilled artisans will appreciate, myelin is made generally by glial cells, but the source of myelin for the peripheral nerves is Schwann cells, while that for the central nervous system is oligodendrocytes. Moreover, the central nervous system (e.g., brain and spinal cord) comprises three distinct layers of the meninges (dura mater, arachnoid mater, and pie mater), while the peripheral nervous system is not surrounded by meningeal layers.

Thus, it will be understood that as used herein, the “nervous system” is not limited to any particular type of structure or cell types and includes neurons (e.g., sensory neurons, motor neurons, and central neurons (or interneurons), ganglia, nerves (i.e., bundles of neurons), as well as any portions thereof such as soma, axons, dendrites, synapses, termini, receptors (e.g., neural receptors), effectors, motor plates, and the like. Also included within the nervous system are nonneuronal cells, such as glial cells, including microglia, astrocytes, oligodendrocytes, ependymal cells, and radial glia in the central nervous system, as well as Schwann cells, satellite cells, and enteric glial cells in the peripheral nervous system. While glial cells lack axons and dendrites characteristic of neurons, and cannot generate action potentials, they nonetheless help modulate neurotransmission. Glia also serve important functions such as surrounding neurons and holding them in place, supplying nutrients and oxygen to neurons, insulating one neuron from its neighbors, destroying pathogens, and removing dead neurons (e.g., during Wallerian degeneration). 036

The term “damage to the nervous system of an animal” means any injury, loss (transient, temporary, short-term, or prolonged), damage, interruption, or the like of a component of the animal's nervous system includes damage to any one or more anatomical, physiological, neurological, biochemical, or other aspect of any part of the entire nervous system, and preferably an aspect of the peripheral nervous system (i.e., any component of the nervous system that is not characterized as the central nervous system, for example, those portions of the nervous system that lack meninges). In addition to discernible damage to the central nervous system and spinal cord, such damage includes damage, however caused, to the cranial or spinal nerves, other nerves, ganglia, individual or groups of neurons, soma, axons, dendrites, synapses, termini, receptors, motor plates, and the like. Damage to glial cells, or any of their functionality is also contemplated as damage to the nervous system.

Aspects of an animal's nervous system that may be evidence of damage thereto include phenotypic changes, in any aspects of either sensory or motor functions, or processing of sensory or motor information. Changes in sensory function or processing include at least changes in mechanoreceptors (e.g., for hearing, balance, stretching), photoreceptors (e.g., for light detection, vision), chemoreceptors (e.g., smell, taste and certain sensors in the digestive and/or circulatory systems), thermoreceptors (temperature), electroreceptors (sensitive to electrical currents in an animal's environment), and/or pain receptors (changes can include desensitivity or hypersensitivity to stimuli, sensation of burning pain, parathesia, and/or acrothesia). Changes in motor receptors can result in increased reflex time (i.e., slowed reflexes), or a decrease in fine motor control, stability, range or extent of movement, and actual or perceived loss of muscle strength, as well as paralysis and/or muscle wasting. Some animals may have receptors that are not known in humans, and such receptors are also contemplated as part of the nervous system for purposes herein, and may be subject to damage. Physical damage (e.g., as discussed above) to the neurons or portions thereof such as axons, dendrites, synapses, etc., or biochemical changes to one or more components of the nervous system that result from disease, metabolic disorders, nutritional deficiencies, and the like are also encompassed within “damage to an animal's nervous system” as used herein. Any disturbance or interruption of, or delay in, the production, storage, release, transmission, reception, propagation, relay, or timing of neurological signals also constitutes “damage.” Skilled artisans will appreciate that common diagnostic tools useful for assessing such damage include neurological exams, computed tomography (CT) scans, magnetic resonance imaging (MRI), electromyography (EMG), nerve conduction tests, such as nerve conduction velocity (NCV) measurements, and biopsy of nerve or skin tissue for direct examination. Damage in a component of an animal's nervous system can be measured relative to a control, a cohort, or relative to an earlier time-point for the same individual in the case of certain types of damage, e.g., progressive loss of function, or chronic damage. Regeneration and/or recovery may also be monitored using any of the foregoing diagnostics or others useful for such purpose. Measurements of recovery may provide an ongoing measure inversely related to damage. Damage to an animal's nervous system may be “physically-induced,” “disease-related,” and/or “systemic/metabolic” as discussed above. Damage to nerves may be classified based on severity as neurapraxia, axonotmesis, and neurotmesis, or as any of Grades I-V.

As used herein, the term “treating, reducing, or preventing” refers to degrees or types of beneficial or therapeutic effect from the compositions and/or methods disclosed herein. In particular, “treating” damage generally indicates that some amount of damage, loss, or decline in the animal's nervous system has already occurred, and a method or composition is useful for ameliorating to some extent, one or more symptoms or results of the associated damage, loss, or decline, i.e., after the fact. “Treating” also indicates that the given composition or method may not be effective at minimizing or short-circuiting any potential damage, loss, or decline. “Reducing” or “decreasing” damage generally indicates that the presence of a composition or use of the method in question is capable of some degree of lessening the damage, loss, or decline before such damage occurs; i.e., it has at least a partial preventative effect. A reducing effect may be observed in terms of the type and/or the extent (e.g., class or grade) of damage or injury. “Preventing” damage indicates that the composition or method in question is capable of preventing one or more results of damage, loss, or decline in an animal's nervous system, i.e., the composition or method is at least partially preventative and may be completely so. “Preventing” may be related to the type or extent of damage, or may also be associated with a delay in onset of damage, loss, or decline (the prevention need not be “permanent” for the animal to benefit, as the prime years may be extended, and/or the quality of life may be enhanced (for example in the case of age-related decline)). Thus, at a minimum an “effective” composition or method is capable of treating damage to provide some positive effect after the fact of damage. Preferably, the composition or method has a reducing effect and/or is capable of at least some preventative effect. More preferably, a composition or method is at least partially capable of fully preventing one or more aspects of damage, loss, or decline that would otherwise likely occur to an animal's nervous system in the absence of the composition or use of the method.

As used herein, the term “food” or “food composition” means a composition that is intended for ingestion by an animal, including a human, and provides nutrition thereto. As used herein, a “food product formulated for human consumption” is any composition specifically intended for ingestion by a human being. “Pet foods” are compositions intended for consumption by pets, particularly by companion animals. A “complete and nutritionally balanced pet food” is one that contains all known required nutrients for the intended recipient or consumer of the food, in appropriate amounts and proportions, based for example on recommendations of recognized authorities in the field of companion animal nutrition. Such foods are therefore capable of serving as a sole source of dietary intake to maintain life or promote production, without the addition of supplemental nutritional sources.

Nutritionally balanced pet food compositions may be a “wet food”, “dry food”, or food of intermediate moisture content. “Wet food” describes pet food that is typically packaged in cans or foil bags, and has a moisture content generally in the range of from about 70 to about 90%. “Dry food” describes pet food that may be similar in nutrient composition to wet food, but contains a limited moisture content. Dry food is typically in the range of from about 5 to about 20%, and therefore may be presented, for example, as small biscuit-like kibbles. In one presently preferred embodiment, the compositions have moisture content from about 5 to about 20%. Dry food products include foods of moisture content in or about the stated range, such that they are substantially resistant to microbial or fungal damage or contamination under normal conditions of storage.

As used herein, a “dietary supplement” is a food that is intended to be ingested in addition to the normal diet of an animal. Dietary supplements may be in any form—e.g., solid, liquid, gel, tablets, capsules, powder, and the like. Preferably, they are provided in convenient dosage forms. In some embodiments, they are provided in bulk consumer packages such as bulk powders or liquids. In other embodiments, supplements are provided in bulk quantities to be included in other food items such as snacks, treats, supplement bars, beverages and the like.

The term “effective amount” means an amount of a compound, material, composition, dietary supplement, medicament, or other material that is effective to achieve a particular biological result, such as reducing, preventing, or treating damage to an animal's vision.

“Young” refers generally to an individual in young adulthood, i.e., matured past puberty or adolescence, as would be defined by species, or by strain, breed or ethnic group within a species, in accordance with known parameters. “Aged” or “old,” as used herein, refers to an individual who is physically or chronologically within about the last 30% of its average life expectancy, as determined by species, or by strain, breed, or ethnic group within a species, in accordance with known parameters. Skilled artisans will appreciate that in general usage “aging” is a process that all living organisms are undergoing, and simply refers to the fact that any living animal is growing older than that animal was previously. As will be clear from the context, the term “aging” as used herein is generally substantially synonymous with “aged” as defined above and thus indicates an animal that is within about the last 30% of its average life expectancy for its kind. 044

As used herein, the “prime years” of an animal's life can extend from young adulthood (“young,” as described above) into the older or “aged” population. Indeed, the prime years of an animal's life can extend essentially until the animal's death, assuming the animal is healthy and active through the animal's older years.

The term “extending the prime” means extending the number of years an animal lives a healthy life and not just extending the number of years an animal lives, e.g., an animal receiving a treatment that extends the prime would be healthy in the prime of its life for a longer time, relative to another animal not receiving the treatment.

The term “health and/or wellness of an animal” means the complete physical, mental, and social well being of the animal, not merely the absence of disease or infirmity.

The term “regular administration” as used herein with respect to the compositions disclosed herein means providing a regular dose of the composition to an animal. Skilled artisans will appreciate that dosing frequency will be a function of the substance that is being administered, and some compositions may require or allow for more or less frequent administration to maintain a desired biochemical, physiological, or gene expression effect, or the like, including neurological and neuroanatomical effects. One goal of regular administration is to provide the animal with a regular and/or consistent dose of the composition or the direct or indirect metabolites that result from such ingestion. Regular and/or consistent dosing will preferably increase blood levels of the components of the compositions or their direct or indirect metabolites compared to those of an animal not receiving administration of the compositions, or even more preferably result in a constant blood level of the those components and/or metabolites. “Regular basis” thus refers to at least monthly administration. “Regular administration” can be once monthly, once weekly, or once daily. Administration can be more frequent than once daily, such as multiple times per day. Administration on other bases is also contemplated, such as every other day, every other week, or every other month, every third day, week, or month, every fourth day, week, or month, and the like. Any dosing frequency, regardless of whether expressly exemplified herein, may be deemed useful for particular applications. The term “extended regular basis” as used herein refers to long term administration of a substance on a regular basis.

“Long term” administration as used herein generally refers to periods in excess of one month. Periods of longer than two, three, or four months are contemplated. Also included are more extended periods that include longer than 5, 6, 7, 8, 9, or 10 months. Periods in excess of 11 months or 1 year are also included. Longer terms use extending over 1, 2, or 3 years or more are also contemplated herein. In the case of certain animals, it is envisioned that the animal would be administered substances identified by the present methods on a regular basis or extended regular basis.

The term “in conjunction with” means that a drug, supplement, food, or other substance is administered to an animal (1) together, e.g., in a composition, particularly a food composition, or (2) separately, e.g., at the same or different time, and/or the same or different frequency, using the same or different administration routes. When administration is “separate” the drug, supplement, food, or other substance can be also given about the same time or periodically. “About the same time” generally means that the substance (e.g., food or drug) is administered at the same time or within about 72 hours of each other. “Periodically” means that the substance is administered on a dosage schedule acceptable for a specific substance. “In conjunction” specifically includes administration schemes wherein substances such as drugs are administered for a prescribed period and compositions of the invention are administered indefinitely. Administration of a composition consistent herewith can be direct or indirect, e.g., in connection with a dietary regimen for the animal. When utilized as a supplement to ordinary dietary requirements, a composition may be administered directly to the animal. The compositions can alternatively be contacted with, or admixed with, daily feed or food, including a fluid such as drinking water, or an intravenous connection for an animal that is receiving such treatment.

As used herein, the term “oral administration” or “orally administering” means that an animal ingests, or a human is directed to feed, or does feed, the animal one or more of the substances described herein. The term “ingestion” is used herein interchangeably with the term “oral administration.” Wherein a human is directed to orally administer or feed the substance, such direction may be that which instructs and/or informs the human that use of the substance may and/or will provide the referenced benefit. Such direction may be oral direction (e.g., through oral instruction from, for example, a physician, veterinarian, or other health professional, or radio or television media (i.e., advertisement), or written direction (e.g., through written direction from, for example, a physician, veterinarian, or other health professional (e.g., prescriptions), sales professional or organization (e.g., through, for example, marketing brochures, pamphlets, or other instructive paraphernalia), written media (e.g., internet, electronic mail, or other computer-related media), and/or packaging associated with the substance.

The term “topical administration” as used herein, means the administration or application of a composition to the skin, mucosa, eye, or any other epithelial surface.

The term “sample” means any animal tissue or fluid containing, e.g., polynucleotides, polypeptides, antibodies, metabolites, and the like, including cells and other tissue containing DNA and RNA. Examples include adipose, blood, cartilage, connective, epithelial, lymphoid, muscle, nervous, sputum, and the like. A sample may be solid or liquid and may be or contain DNA, RNA, cDNA, tissue(s), bodily fluids such as blood or urine, cells, cell preparations or fractions thereof, chromosomes, organelles, and the like.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof. A single package may be containers of individual food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

All percentages expressed herein are by weight of the composition on a dry matter (or “dry weight”) basis unless specifically stated otherwise. Skilled artisans will appreciate that the terms “dry matter basis” or “dry weight basis” mean that the amount of the ingredient present in the composition is expressed relative to the composition after the free moisture in the composition is removed.

Dosages expressed herein are generally indicated as milligrams or grams per kilogram of body weight (mg/kg or g/kg) unless expressed otherwise.

As used herein, ranges are used herein in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a compound” or “a method” includes a plurality of such “compounds” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context.

The terms “comprising” or “including” are intended to include embodiments encompassed by the terms “consisting essentially of” and “consisting of”. Similarly, the term “consisting essentially of” is intended to include embodiments encompassed by the term “consisting of”.

The methods and compositions and other advances disclosed here are not limited to particular methodology, protocols, and reagents described herein because, as skilled artisans will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### The Invention

In one aspect, the invention provides methods for treating, reducing, or preventing damage to the nervous system of animals. The methods comprise identifying an animal susceptible to or suffering from damage to at least one component of the nervous system and administering in conjunction to the animal one or more unsaturated fatty acids (UFA) and one or more nitric oxide releasing compounds (NORC) in an amount effective for treating, reducing, or preventing damage to the nervous system of the animal.

The invention is based in part upon the surprising discovery that UFA and NORC are useful for treating, reducing, or preventing damage to the nervous system of animals, particularly in combination with B vitamins and/or antioxidants. The damage can be caused by injury, disease, systemic conditions, metabolic conditions, aging, or other mechanism. In particular, and as described in greater detail below and in the examples, methods have been developed based on administering such compounds to animals whereby damage to one or more aspects of the animal's nervous system is treated, reduced, prevented, or even reversed. The results are surprising in view of reports regarding the role of NO and of iNO synthase enzymes in certain forms of damage to the nervous system, including reports of the role of NO in inducing, promoting, sustaining, or mediating nerve damage in certain neuropathies.

In various embodiments, the methods are useful for treating, reducing, or preventing damage to at least one component of the nervous system, e.g., the brain, spinal cord, and peripheral nervous system. In certain embodiments, the methods comprise administering to an animal a composition comprising one or more UFA and one or more NORC in amounts sufficient for treating, reducing, or preventing damage to a component of the nervous system of the animal.

In certain embodiments, the methods are useful when damage to the nervous system is caused by damage to any portion or component of an animal's nervous system, e.g., the brain, the spinal cord, a peripheral nerve, a glial cell, a ganglion, myelin, or a neuron.

In one embodiment, the damage is to a neuron, or at least a portion of a neuron that is soma, axon, dendrite, synapse, terminus, receptor, or effector. In another embodiment, damage is to a motor plate. In other embodiments, the damage is to a glial cell such as a microglia, astrocyte, oligodendrocyte, ependymal cell, radial glia, Schwann cell, satellite cell, enteric glial cell, or other glial cell. Damage to a glial cell comprises any damage to a function or structure of a glial cell, for example, any alteration of myelin production, or alteration of any neural support function of a glial cell.

In certain preferred embodiments, the methods are applicable when the damage is to a component of the animal's brain, especially traumatic damage. Traumatic brain damage is fairly common and a major cause of death or disability in humans and other animals.

In other embodiments, the methods are applicable wherein the damage is to a component of the animal's spinal cord, especially traumatic damage. Traumatic spinal cord damage is fairly common, and a major cause of death or disability in humans and animals.

In further embodiments, the methods are applicable when the damage is to a component of the animal's peripheral nervous system. Peripheral nervous system damage is fairly common and tends to be more amenable to repair, regeneration, or recovery than damage to central nervous system. In one such embodiment, damage may be to any component or portion of the peripheral nervous system, such as a neuron or portion thereof or to a glial cell. A damaged glial cell in the peripheral nervous system can be a Schwann cell, satellite cell, enteric cell, or other glial cell of the peripheral nervous system.

The methods can be applied and are useful to any type or degree of damage to an animal's nervous system. Thus, the damage can be of any class or type according to known classification systems, such as Seddon's classification, or Sunderland's grading system. As classified in Seddon's system, the damage can be considered as neurapraxia, axonotmesis, or neurotmesis for purposes herein. Using Sunderland's system, damage considered as any of Grades I-V is useful. Independent of the amount of damage, the animal to whom the compounds or compositions are administered benefits in one or more ways from receiving the compounds or compositions. For example, the animal receiving the compounds or compositions may have less total damage, improved survival, improved overall health and/or quality of life, a faster rate of recovery, a further extent of recovery, or the like in any one or more observable parameters related to the damage to the nervous system. Even in an animal with severe damage, the methods, compounds, and compositions disclosed herein will provide observable, ascertainable, or measurable benefit to the animal.

In one embodiment, the nervous system damage is caused by physical injury, disease, systemic status of the animal, metabolic status of the animal, or combination thereof such as any of those set forth in the definitions provided above. The damage is a peripheral neuropathy in one presently preferred embodiment. In various preferred embodiments, the damage is associated with normal aging, trauma, the use of a prescription drug, non-prescription drug, diabetes or metabolic syndrome, or an acute or chronic deficiency in one or more nutrients. In other embodiments, the damage is associated with an infectious and/or inflammatory disease, or with an in-born error or metabolism or other error or mutation in an animal's genetic material.

Regardless of the cause of the damage to the nervous system, one or more effects of such damage can be observed, ascertained, measured, or quantified. Skilled artisans will understand the established methods for diagnosing such damaged states, and/or inspecting for known signs of such neurological or other nervous system injuries. In addition, the literature is replete with information on measuring damage or the like to components of the nervous systems of animals.

Thus, the damage in certain embodiments is caused by normal aging, such that there appears to be an absence of an injury or disease that is identifiable as a substantial source of the damage. In other embodiments, the damage is caused by disease. Included among the diseases of interest herein are various infectious, inflammatory, genetic and/or neoplastic diseases that impact the nervous system directly or indirectly to cause damage. Such diseases can occur in animals of any age, but may occur with greater likelihood and/or frequency in older animals than in the young, for example because the immune system in an older animal may not be as proficient in warding off invading infections. Older animals may also tend to be more susceptible to inflammation, as well to forms of diabetes, metabolic syndrome, neoplastic diseases, and the like. Examples of such diseases are found in the definitions section supra.

Some diseases that can damage the nervous system may be specific to certain animals, e.g., a certain species or type of animal, such as companion animals, e.g., dogs and/or cats. Some of the diseases are listed generically, i.e., there may be many types of herpesvirus diseases, many types of genetic diseases, and many types of peripheral neuropathy. Thus, some of the diseases are not caused by one specific etiologic agent, but are more descriptive of the type of disease or the result. Many of the diseases that can cause damage to one or more components of the nervous system can have both primary (direct) and secondary, as well as even more remote effects on an animal's nervous system, independent of the animal's age.

The methods in various embodiments are directed to humans or companion animals, such as dogs and cats. The animal can be of any age, but the methods are also well-suited to use with aged (i.e., aging) animals because of the propensity of aging animals to suffer age-related damage or decline in at least one component of their nervous system, for example, the damage to a component of the peripheral nervous system. In some embodiments, age-related damage of the nervous system comprises damage of one or more of a glial cell, a ganglion, myelin, a neuron, or another component of the nervous system. In other embodiments, age-related damage is to at least a portion of a neuron that is soma, axon, dendrite, synapse, terminus, receptor, or effector, or to a motor plate. The glial cell is a Schwann cell, satellite cell, enteric cell, or other glial cell of the peripheral nervous system in certain embodiments.

The UFA useful in the invention are any type or from any source. In particular embodiments, the UFA are one or more of ALA, EPA, DPA, DHA, or another n-3 fatty acid from any source, e.g., natural or synthetic. Fish oils are well known and popular sources of UFA, particularly long-chain polyunsaturated fatty acids (LCPUFA) for use in foods and supplements.

The UFA are administered to the animal in amounts of from about 0.001 to about 50 g/kg, although greater or lesser amounts can be administered. In various embodiments, the UFA are administered in amounts of from about 0.001 to about 25 g/kg, from about 0.001 to about 10 g/kg, or from about 0.001 to about 5 g/kg. Preferably, the UFA are administered in amounts of from about 0.001to about 1 g/kg, more preferably from about 0.001 to about 0.5 g/kg. When administered in a composition, the composition comprises from about 0.1 to about 50% UFA. More preferably, the UFA content is from about 0.5 to about 20%, from about 1 to about 10%, or from about 2 to about 5%. In some embodiments, the acceptability of the composition from a sensory perspective may decrease as the content of the UFA goes up, and thus at high concentrations, sensory attributes such as flavor or aftertaste may be considered in formulating the compositions.

The NORC useful in the invention are any type from any source. In particular, embodiments, the NORC are arginine or a nitric oxide-releasing analog or derivative of arginine. In other embodiments, citrulline or ornithine are used as a source of NORC.

The NORC are administered to the animal in amounts of from about 0.001 to about 50 g/kg, although greater or lesser amounts can be administered. In various embodiments, the NORC are administered in amounts of from about 0.001 to about 25 g/kg, from about 0.001 to about 10 g/kg, or from about 0.001 to about 5 g/kg. Preferably, the NORC are administered in amounts of from about 0.001 to about 1 g/kg, more preferably from about 0.001 to about 0.5 g/kg. When administered in a composition, the composition preferably comprises from about 0.1 to about 20% NORC. Other compositions may comprise, for example, from about 0.5 to about 15%, from about 1 to about 10%, or from about 2 to about 5% NORC.

In various embodiments, the methods for using UFA and NORC for treating, reducing, or preventing damage to the nervous system of an animal further comprise administering in conjunction to the animal one or more B vitamins, one or more antioxidants, or a combination of B vitamins and antioxidants in an amount effective for treating, reducing, or preventing the damage. In preferred embodiments, the methods further comprise administering in conjunction to the animal one or more B vitamins and one or more antioxidants in an amount effective for treating, reducing, or preventing the damage. Generally, the B vitamins are administered in an amount of from about 0.1 to about 40 times the recommended daily requirement of B vitamins, preferably from about 0.5 to about 20 times the recommended daily requirement of B vitamins, and the antioxidants are administered in an amount of from about 0.1 to about 10 times the recommended daily allowance for the antioxidants, preferably from about 0.5 to about 5 times the recommended daily allowance. When administered in a composition, the composition preferably comprises from about 0.1 to about 40 times the recommended daily requirement of B vitamins and from about 0.1 to about 10 times the recommended daily allowance for the antioxidants.

In various embodiments, the methods are useful to treat damage of the nervous system of an animal, e.g., to help mitigate one or more aspects of damage after it has occurred. In preferred embodiments, the methods are useful to reduce the damage than would otherwise occur in the absence of such methods. More preferably, the methods are useful for preventing some, substantially all or all damage to the nervous system of an animal. Other preferred embodiments feature methods that delay the onset of one or more aspects of damage, wherein the longer the delay to onset, the more preferred the embodiment. Still other embodiments feature methods that can partially or fully reverse one or more aspects of the damage of the nervous system. Skilled artisans will appreciate that the longer damage has gone untreated, the more difficult is the treating, decreasing, or prevention of that or other damage to the nervous system. Preferably, the methods are applied to at-risk animals, e.g., animals with diseases that may cause damage to the nervous system, aging animals prior to the damage of the nervous system, or at a time when damage or decline has not onset, is minimal, or is recently onset.

The compositions for use herein optionally comprise one or more supplementary substances that contribute to the observed effect of treating, reducing, or preventing damage to a component of the nervous system, or which promote or sustain general health and wellness, as would be appreciated by skilled artisans. The compositions may thus further comprise substances such as minerals, other vitamins, salts, functional additives including, for example, palatants, colorants, emulsifiers, antimicrobial or other preservatives. Minerals that may be useful in such compositions include, for example, calcium, phosphorous, potassium, sodium, iron, chloride, boron, copper, zinc, magnesium, manganese, iodine, selenium, and the like. Examples of additional vitamins useful herein include such fat-soluble vitamins as A, E, and K. Inulin, amino acids, enzymes, coenzymes, and the like may be useful to include in various embodiments.

In certain embodiments, compositions comprise UFA and NORC. In others, compositions consist essentially of UFA and NORC. In yet others, compositions consist of UFA and NORC. In various embodiments, the compositions comprise, consist essentially of, or consist of UFA and NORC and either B vitamins, antioxidants, or a combination thereof.

Any antioxidant suitable for administration to an animal is contemplated for use herein. Antioxidants are well known in the art of food technology and food formulation. Natural antioxidant compounds include the antioxidant vitamins (such as A, C and E, and derivative, conjugates, or analogs thereof). Compounds such as α-lipoic acid, chlorophyll and derivatives thereof, glutathione, ubiquinols (e.g., coenzyme Q10), carotenoids (e.g., lycopene), flavonoids, phenolics, and polyphenols, and pycnogenol are known to be excellent antioxidants, and most can be derived from one or more plant sources. Many plant extracts, including extracts from flowers, fruit, vegetables, herbs, seeds, bark, stems, shoots, roots and/or other parts of plants are known to contain useful antioxidants. Specific examples of plant sources of antioxidants include fruits such as berries (e.g., elderberry, cherry, blackberry, strawberry, raspberry, cranberry, crowberry, blueberry, bilberry/wild blueberry, black currant), pomegranate, grape, orange, plum, pineapple, kiwi fruit, citrus (including, for example, lemon and grapefruit), dried fruits like apricots, prunes, and dates; and vegetables, such as cruciferous vegetables (for example kale, cabbage, Brussels sprouts, broccoli, and bok choy), parsley, artichoke, spinach, ginger, garlic, beets, peppers (including chili and other ‘hot’ peppers). Also good sources of plant antioxidants are nuts and seeds such as pecans, walnuts, hazelnuts, ground nuts, and sunflower seeds, grape seeds; legumes, including soy, broad, and pinto beans for example; cereals such as barley, millet, oats, and corn. Natural antioxidants are also derived from a wide variety of spices including cloves, cinnamon, rosemary, and oregano. Less widely known sources of antioxidants include Ginkgo biloba, and tropical plants such as uyaku, and carica papaya. Certain other antioxidants have become of great interest in recent years and would be suitable for use herein, including those from various fermented and unfermented teas and green tea, fermented products such as red wine, and so-called “superfruits” such as noni, mangosteen, acai, mango, goji, sea-buckthorn, and others. Selenium is an excellent oxygen scavenger and works well, especially with vitamin E compounds and/or related tocopherol and/or tocotrienol compounds. Synthetic dietary antioxidants include butylated hydroxyanisole (BHA) and butylated hydroxytoluene (BHT) which are commonly used in food products. Any of the foregoing, alone or in combination, are suited for use herein, as are combinations of natural and synthetic antioxidants. In one embodiment, the antioxidants comprise astaxanthin alone or in combination with other antioxidants.

Preferred antioxidants include one or more of the antioxidant vitamins (e.g., A, C, and E), or a tocopherol or tocotrienol compound that has similar or better antioxidant properties and preferably also has vitamin E activity. Other preferred antioxidants include zeaxanthin, astaxanthin, lutein, or selenium. Selenium is particularly useful when vitamin E compounds (including tocopherols and tocotrienols) are present in the formulation.

In various embodiments, the UFA, NORC, B vitamins, and/or antioxidants are administered to the animal on a long-term basis, preferably on an extended regular basis. Preferably, the UFA, NORC, B vitamins, and/or antioxidants are administered to the animal on a regular basis, preferably daily. 088

The methods provided are generally based on using compositions that may readily be formulated as a human food composition, a pet food composition, or a dietary supplement. Such compositions include foods intended to supply the necessary dietary requirements for a human, or a companion animal, or as animal treats (e.g., biscuits), or dietary supplements. The formulation of such compositions is readily understood by skilled artisans who will appreciate that the food compositions may further comprise protein, fat, moisture, for example, from about 15 to about 50% protein, from about 5 to about 40% fat, and a moisture content of from about 5 to about 20%. Such compositions may have from about 5 to about 10% ash content. Also, as described in greater detail below, the composition can contain additional ingredients, including vitamins, minerals, prebiotics, probiotics, or a combination thereof.

Certain aspects of the invention are preferably used in combination with a complete and balanced food. According to certain embodiments, the compositions comprising the UFA and NORC, and B vitamins and antioxidants if needed, are preferably used with or formulated into a complete and balanced commercial food. The compositions and dietary supplements may also be specially formulated for the intended recipients or consumers, such as for adult animals or for older or young animals. For example, a composition adapted for aging animals can be prepared, as can compositions adapted for the nutritional needs of active, pregnant, or lactating animals, or even, for example, for puppies or kittens. In general, specialized compositions will comprise energy and nutritional requirements appropriate for animals at different stages of development or age, or of different health or nutritional status. 090

In one embodiment, the composition is formulated as a refrigerated or frozen composition. In other embodiments, the composition may be a dry composition (e.g., kibble), semi-moist composition, wet composition, or any mixture thereof. In another embodiment, the composition is a dietary supplement formulated as a gravy, drink, beverage, yogurt, powder, granule, paste, suspension, chew, morsel, treat, snack, pellet, pill, capsule, tablet, or any other suitable delivery form. The dietary supplement can comprise a high concentration of the UFA and NORC such that the supplement can be administered to the animal in small amounts, or in the alternative, can be diluted prior to administration to an animal. The dietary supplement may require admixing, or preferably be admixed with water or other diluent prior to administration to the animal. 091

In various embodiments, pet food or pet treat compositions that contain UFA and NORC, and B vitamins and antioxidants if needed, comprise from about 15 to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof. The compositions may further comprise from about 5 to about 40% fat. The compositions may further comprise a source of carbohydrate. The compositions may comprise from about 15 to about 60% carbohydrate. Examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products. In some embodiments, the ash content of the composition ranges from less than 1 to about 15%, preferably from about 5 to about 10%. The moisture content can vary depending on the desired nature of the composition. In a preferred embodiment, the composition is a complete and nutritionally balanced pet food. In this embodiment, the pet food may be a “wet food”, “dry food”, or food of intermediate moisture content. Presently preferred are dry food compositions that are extruded food products, such as pet foods, or snack foods for either humans or companion animals. The compositions may also comprise one or more fiber sources. The term “fiber” includes all sources of “bulk” in the food whether digestible or indigestible, soluble or insoluble, fermentable or nonfermentable. Preferred fibers are from plant sources such as marine plants but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, fructooligosaccharide, pectin, short chain oligofructose, mannanoligofructose, soy fiber, arabinogalactan, galactooligosaccharide, arabinoxylan, or mixtures thereof. Alternatively, the fiber source can be a fermentable fiber. Fermentable fiber has previously been described to provide a benefit to the immune system of a companion animal. Fermentable fiber and other compositions can act as prebiotics (as discussed below) to enhance the growth of probiotic organisms in the gastrointestinal tract.

In various embodiments, the methods further comprise administering in conjunction with the UFA and NORC ( and optional B vitamins, antioxidants, or combinations thereof) at least one of (1) one or more probiotics; (2) one or more inactivated probiotics; (3) one or more components of inactivated probiotics that promote health benefits similar to or the same as the probiotics; (4) one or more prebiotics; and (5) combinations thereof. When administered with compositions, the probiotics or their components can be integrated into the compositions (e.g., uniformly or non-uniformly distributed in the compositions) or applied to the compositions (e.g., topically applied with or without a carrier). Such methods are known to skilled artisans, e.g., U.S. Pat. No. 5,968,569 and related patents.

Typical probiotics include, but are not limited to, probiotic strains selected from Lactobacilli, Bifidobacteria, or Enterococci, e.g., Lactobacillus reuteii, Lactobacillus acidophilus, Lactobacillus animalis, Lactobacillus ruminis, Lactobacillus johnsonii, Lactobacillus casei, Lactobacillus paracasei, Lactobacillus rhamnosus, Lactobacillus fermentum, and Bifidobacterium sp., Enterococcus faecium and Enterococcus sp. In some embodiments, the probiotic strain is selected from the group consisting of Lactobacillus reuteri (NCC2581; CNCM I-2448), Lactobacillus reuteri (NCC2592; CNCM I-2450), Lactobacillus rhamnosus (NCC2583; CNCM I-2449), Lactobacillus reuteri (NCC2603; CNCM I-2451), Lactobacillus reuteri (NCC2613; CNCM I-2452), Lactobacillus acidophilus (NCC2628; CNCM I-2453), Bifidobacterium adolescentis (e.g., NCC2627), Bifidobacterium sp. NCC2657 or Enterococcus faecium SF68 (NCIMB 10415). The probiotics are administered in amounts sufficient to supply from about 10⁴ to about 10¹² cfu/animal/day, preferably from 10⁵ to about 10¹¹ cfu/animal/day, most preferably from 10⁷ to 10¹⁰ cfu/animal/day. When the probiotics are killed or inactivated, the amount of killed or inactivated probiotics or their components should produce a similar beneficial effect as the live microorganisms. Many such probiotics and their benefits are known to skilled artisans, e.g., EP1213970B1, EP1143806B1, U.S. Pat. No. 7,189,390, EP1482811B1, EP1296565B1, and U.S. pAT. nO. 6,929,793. In a preferred embodiment, the probiotic is Enterococcus faecium SF68 (NCIMB 10415). In one embodiment, the probiotics are encapsulated in a carrier using methods and materials known to skilled artisans.

As stated, the methods may use one or more prebiotics, e.g., fructo-oligosaccharides, gluco-oligosaccharides, galacto-oligosaccharides, isomalto-oligosaccharides, xylo-oligosaccharides, soybean oligosaccharides, lactosucrose, lactulose, and isomaltulose. Fructo-oligosaccharides are found naturally in many foods such as wheat, onions, bananas, honey, garlic, and leeks. In one embodiment, the prebiotic is chicory root, chicory root extract, inulin, or combinations thereof. Generally, prebiotics are administered in amounts sufficient to positively stimulate the healthy microflora in the gut and cause these “good” bacteria to reproduce. Typical amounts are from about one to about 10 grams per serving or from about 5 to about 40% of the recommended daily dietary fiber for an animal. The amount of prebiotic can be determined by skilled artisans based upon (1) the type and nature of the prebiotic and the type and nature of the desired composition and (2) the type and nature of the animal that will consume the prebiotics, e.g., the animal's age, weight, general health, sex, gut microflora status (including presence of harmful bacteria), and diet.

The probiotics and prebiotics can be made part of a composition by any suitable means. Generally, the agents are mixed with the composition or applied to the surface of the composition, e.g., by sprinkling or spraying. When the agents are part of a kit, the agents can be admixed with other materials or in their own package. Typically, the food composition contains from about 0.1 to about 10% prebiotic, preferably from about 0.3 to about 7%, most preferably from about 0.5 to 5%, on a dry matter basis. The prebiotics can be integrated into the compositions using methods known to skilled artisans, e.g., U.S. Pat. No. 5,952,033.

The probiotics may be administered in any effective amount for any duration, whether short or preferably longer term, and at any useful frequency. In one embodiment, the methods employ compositions that are administered on an extended regular basis, preferably on a daily basis. According to the methods of the invention, administration of the compositions, including administration as part of a dietary regimen, can span a period ranging from parturition through the adult life of the animal.

In certain embodiments, the animal is a young or growing animal. In other embodiments, the animal is an adult or mature animal. In other embodiments, the animal is an aging animal. An animal that has reached about 30% of its projected lifespan is generally suitable. In certain embodiments, administration begins, for example, on a regular or extended regular basis, when the animal has reached more than about 30, 40, or 50% of its projected or anticipated lifespan. In some embodiments, the animal has attained 40, 45, or 50% of its anticipated lifespan. In yet other embodiments, the animal is older having reached 60, 66, 70, 75, or 80% of its likely lifespan. A determination of lifespan may be based on actuarial tables, calculations, estimates, or the like, and may consider past, present, and future influences or factors that are known to positively or negatively affect lifespan. Consideration of species, gender, size, genetic factors, environmental factors and stressors, present and past health status, past and present nutritional status, stressors, and the like may also influence or be taken into consideration when determining lifespan.

The methods described herein may also employ pharmaceutical or nutraceutical compositions, formulated for administration by any selected route, as described in greater detail below.

In some embodiments, the compounds or compositions of the invention are administered to the animal in conjunction with one or more therapeutic agents useful for, and in an amount effective for, treating, reducing, or preventing damage of one or more components of the nervous system of an animal. Therapeutic agents useful for treating, reducing, or preventing damage to an animal's nervous system includes one or more of a corticosteroid, erythropoietin, an immunosuppressive drug, mexiletine, an immunoglobulin, a therapeutic antibody, a therapeutic cell, retinoic acid or a derivative thereof, a microRNA, a neurotrophin, an antidepressant drug, an antiepileptic drug, sodium valproate or a pharmaceutically acceptable salt of valproic acid, a cannabinoid, an anticonvulsant, and lidocaine.

Skilled artisans will appreciate that some of the foregoing represent classes of therapeutic agents and any member of the class may be suitable for use herewith. In presently preferred embodiments, the corticosteroid is methylprednisolone; the immunosuppressive drug is for example, one or more of prednisone, cyclosporine, or azathioprine; the neurotrophin is NTF-3; the antidepressant drug is amitriptyline or other tricyclic antidepressant; the antiepileptic drug is gabapentin, phenytoin, or carbamazepine; and/or the anticonvulsant is pregabalin. Where a therapeutic cell is employed, it is preferably a stem cell, or a cell capable of producing a cell product that is useful for the damaged nervous system component.

The composition administered in the methods is a pharmaceutical or nutraceutical composition in certain embodiments, and optionally comprises one or more of the foregoing agents useful for treating, reducing, or preventing the damage to a nervous system component.

In another embodiment, the invention provides pharmaceutical compositions comprising a composition of the invention as described above, comprising at least UFA and NORC, and one or more pharmaceutically acceptable carriers, diluents, or excipients. Generally, pharmaceutical compositions are prepared by admixing a compound or composition with excipients, buffers, binders, plasticizers, colorants, diluents, compressing agents, lubricants, flavorants, moistening agents, and the like, including other ingredients known to skilled artisans to be useful for producing pharmaceuticals and formulating compositions that are suitable for administration to an animal as pharmaceuticals.

The pharmaceutical composition can be formulated for any mode of administration. In one embodiment, the pharmaceutical composition is formulated for oral administration. In another embodiment, the composition is formulated for topical administration. Suitable topical formulations may include solutions, emulsions, creams, ointments, gels, liposomes, biodegradable microparticles, and other such delivery vehicles as would be well understood by the pharmaceutical chemist.

In other embodiments, the compounds or compositions are administered to the animal in conjunction with one or more physical treatments useful for treating, reducing, or preventing the damage to a nervous system component. Again, preferably in such methods the damage of the nervous system comprises damage to a component of the peripheral nervous system, such as one or more of a glial cell, a ganglion, myelin, a neuron, or another component of the nervous system, as described herein above.

Where the methods involve administering the composition to the animal in conjunction with one or more physical treatments, in various embodiments the treatment comprises one or more of exercise, plasmapheresis, orthopedic supports or braces, and/or TENS (Transcutaneous Electrical Nerve Stimulation).

Damage to a portion of a neuron that is soma, axon, dendrite, synapse, terminus, receptor, or effector, is contemplated herein as amenable to the methods provided. Animals having damage to a motor plate may also benefit from the methods and compositions.

In addition to neuron damage, glial cell damage, e.g., to Schwann cells, satellite cells, enteric cells, or other glial cells of the peripheral nervous system may be treated, reduced, or prevented using the methods and compositions provided.

Such physical treatments can be administered further in conjunction with one or more therapeutic agents also useful for treating, reducing, or preventing the damage. Each such therapeutic agent may be used in accordance with the present methods, alone or in combination, at any suitable dose.

In another aspect, the invention provides kits suitable for treating, reducing, or preventing damage to at least one component of the nervous system of an animal. The kits comprise in separate containers in a single package or in separate containers in a virtual package, as appropriate for the kit component, one or more UFA and one or more NORC. In various embodiments, the kits further comprise one or more of (1) one or more other ingredients suitable for consumption by an animal; (2) one or more B vitamins; (3) one or more antioxidants; (4) one or more therapeutic agents useful for treating, reducing, or preventing damage to an animal's nervous system; (5) one or more prebiotics; (6) one or more probiotics; (7) one or more diagnostic devices suitable for determining whether an animal could benefit from methods and compositions for treating, reducing, or preventing damage to at least one component of the nervous system; (8) instructions for how to combine or prepare the UFA, NORC, and any other ingredients provided in the kit for administration to an animal; (9) instructions for how to use one or more of the kit components in combination, prepared, or otherwise for the benefit of an animal; and (10) a device for administering the combined or prepared kit components to an animal.

In one embodiment, the kits contain the UFA and NORC in a composition and contains one or more of the other kit components. In other embodiments, the kits contain the UFA and NORC and one or more of B vitamins and antioxidants in a composition and contains one or more of the other kit components. In a preferred embodiment, the kit contains the UFA, NORC, B vitamins, and antioxidants in a composition and contains one or more of the other kit components. In preferred embodiments, the composition is a food composition suitable for consumption by an animal.

The kits are particularly useful where the damage of the nervous system comprises damage of one or more components of the peripheral nervous system. Such damage includes damage to one or more of a glial cell, a ganglia, myelin, neurons, nerves, or another component of the nervous system. Such damage is described more fully above regarding the first aspect of the invention, and in the definitions sections herein. In one embodiment, the damage is to at least a portion of a neuron that is soma, axon, dendrite, synapse, terminus, receptor, or effector. The damage is to a motor plate in yet other embodiments. Glial cells of the peripheral nervous system comprise, for example Schwann cells, satellite cells, enteric cells, and other glial cells.

In another aspect, the invention provides means for communicating information about or instructions for one or more of (1) using the disclosed methods or compositions for treating, reducing, or preventing damage to at least one component of the nervous system; (2) admixing the UFA, NORC, B vitamins, antioxidants, or other components disclosed herein to produce a composition suitable for treating, reducing, or preventing damage to a component of the nervous system in the animal; (3) using the disclosed kits for treating, reducing, or preventing damage to a component of the nervous system in the animal; or (4) administering the compositions to an animal; the means comprising one or more of a physical or electronic document, digital storage media, optical storage media, audio presentation, audiovisual display, or visual display containing the information or instructions.

The means is preferably a displayed website, a visual display kiosk, a brochure, a product label, a package insert, an advertisement, a handout, a public announcement, an audiotape, a videotape, a DVD, a CD-ROM, a computer-readable chip, a computer-readable card, a computer-readable disk, a USB device, a FireWire device, a computer memory, or any combination thereof.

In another aspect, the invention provides methods of manufacturing a food composition comprising UFA, NORC, and one or more ingredients suitable for consumption by an animal. The methods comprise admixing one or more ingredients suitable for consumption by an animal with UFA and NORC. Alternatively, UFA and NORC can be applied separately or in combination onto the food composition, e.g., as a coating or topping. The UFA and NORC can be added at any time during the manufacture and/or processing of the food composition. This includes, for example, admixing the UFA and NORC as part of the core formulation of the “body” of the food composition or applying them as a coating, i.e., primarily to the surface of the food composition after its manufacture. The compositions can be made according to any method suitable in the art.

The ingredients suitable for consumption by an animal are preferably one or more B vitamins, and/or one or more antioxidants. The inclusion of B vitamins and antioxidants are discussed more fully with respect to other aspects of the invention, and identical considerations apply to the methods of manufacture. Other ingredients may be included in the methods, including protein, carbohydrate, fat, moisture, fiber, pre- and probiotics, and the like. Preferred ingredients include any ingredients that promote or sustain health of an animal, or ingredients, particularly functional food ingredients that support the nervous system, or can aid in the repair of damage of the nervous system. The food compositions may be of any type or kind, and intended for consumption by any animal.

Preferably, the UFA, NORC, B vitamins, or antioxidants are in the food composition in an amount effective for treating, reducing, or preventing damage to the nervous system of an animal when the food is administered to the animal in at least a recommended amount.

In another aspect, the invention provides packages. The packages comprise one or more of UFA, NORC, B vitamins, and antioxidants in an amount useful for treating, reducing, or preventing damage to at least one component of the nervous system and a label affixed to the package containing a word or words, picture, design, symbol, acronym, slogan, phrase, or other device, or combination thereof, that indicates that the contents of the package contains one or more compounds suitable for treating, reducing, or preventing damage to the nervous system of an animal. In various embodiments, the compounds are contained in a composition, e.g., a food composition or a pharmaceutical or nutraceutical composition.

In another aspect, the invention provides packages comprising one or more of UFA, NORC, B vitamins, and antioxidants and a label affixed to the package containing a word or words, picture, design, symbol, acronym, slogan, phrase, or other device, or combination thereof, that indicates that the contents of the package contains compounds or compositions suitable for treating, reducing, or preventing damage to a component of the nervous system of an animal.

In another aspect, the invention provides medicaments and uses therefor. The medicaments comprise UFA and NORC. Thus, the invention provides for the use of UFA and NORC to prepare a medicament for treating, reducing, or preventing damage to at least one component of the nervous system of an animal. Preferably, the animal is a human, or a companion animal.

In certain embodiments, the medicament further comprises one or more B vitamins, one or more antioxidants, or combination thereof. As with other aspects of the invention, the medicament preferably comprises a NORC that is arginine, a nitric oxide-releasing derivative or analog of arginine, citrulline, or ornithine. The use of the medicament is preferred in embodiments where the damage is to a component of the animal's brain, especially traumatic damage. An animal subject to damage to any portion of a neuron such as soma, axon, dendrite, synapse, receptor, or effector can preferably benefit from use of the methods, compositions, kits, medicaments and the like provided herein. The amount of UFA, NORC, B vitamins, and antioxidants used in the medicament is the same as the amount of such compounds given herein for the methods of the invention. The use of the medicament is also preferred in embodiments where the damage is to a component of the animal's spinal cord, especially traumatic damage. An animal subject to damage to any portion of a neuron such as soma, axon, dendrite, synapse, receptor, or effector can preferably benefit from use of the methods, compositions, kits, medicaments, and the like provided herein. The use of the medicament is also preferred in embodiments where damage of the nervous system is to a component of the peripheral nervous system, such as glial cells, ganglia, myelin, neurons, or some other component. An animal subject to damage to any portion of a neuron such as soma, axon, dendrite, synapse, terminus, receptor, or effector, or damage to a motor plate can preferably benefit from use of the methods, compositions, kits, medicaments and the like provided herein. In one embodiment, a glial cell of the peripheral nervous system is a Schwann cell, satellite cell, enteric cell, or other glial cell.

The compounds and compositions of the invention, including the pharmaceutical compositions and medicaments, are administered to the animal using a variety of administration routes. Such routes include oral, intranasal, intravenous, intramuscular, intragastric, transpyloric, subcutaneous, rectal, and the like. Preferably, the compounds and compositions are administered orally.

In another aspect, the invention provides methods for treating, reducing, or preventing damage to at least one component of the nervous system of an animal. The methods comprise identifying an animal for which treating, reducing, or preventing damage to at least one component of the nervous system is desired and administering in conjunction to the animal one or more UFA and one or more supplements that can be metabolized by the animal to produce NORC in an amount effective for treating, reducing, or preventing damage to at least one component of the nervous system of the animal.

For such methods, the supplement is generally citrulline or ornithine, as arginine and most of its NORC derivatives do not require metabolism to produce a NORC compound. In one embodiment, the animal is a human. In another, the animal is a canine or a feline companion animal. In various embodiments, the animal is an aged animal.

The UFA generally comprises one or more of ALA, EPA, DPA, DHA, or another n-3 fatty acid from any source, but a preferred UFA is from a fish oil source in certain embodiments.

In some embodiments, the UFA and supplement are administered together or separately in a composition. Generally, the composition comprises from about 0.1 to about 50% UFA, and in some embodiments, the UFA content is from about 0.5 to about 20%, from about 1 to about 15%, or from about 1 to 2 to about 5 to 10%. Similarly, the composition comprises from about 0.1 to about 20% supplement.

In various embodiments, the composition further comprises one or more B vitamins in an amount effective for treating, reducing, or preventing damage to at least one component of the nervous system of an animal, such as from about 0.1 to about 40 times the recommended daily requirement of B vitamins.

In some embodiments, the composition further comprises one or more antioxidants in an amount effective for treating, reducing, or preventing damage to at least one component of the nervous system of an animal, such as from about 0.0001 to about 25% of antioxidants.

In some embodiments, the composition comprises both antioxidants and one or more B vitamins in an amount effective for treating, reducing, or preventing damage to the nervous system of an animal. For example, the composition comprises from about 0.1 to about 40 times the recommended daily requirement of B vitamins and from about 0.0001 to about 25% of antioxidants in certain embodiments.

Methods in accordance with this aspect use such compositions as described above and throughout this disclosure, and are preferably useful where the damage of the nervous system comprises damage of one or more components of the peripheral nervous system, including for example a glial cell, a ganglion, myelin, a neuron, or another component of the nervous system. In one embodiment, damage to at least a portion of a neuron that is soma, axon, dendrite, synapse, terminus, receptor, or effector, or damage to a motor plate can be treated, reduced, or prevented. In another embodiment, the glial cell is a Schwann cell, satellite cell, enteric cell, or other glial cell of the peripheral nervous system.

In any such methods, the composition may be formulated as a human food composition, pet food composition, or a dietary supplement for some embodiments. In addition to the UFA and the NORC, the food composition further can comprise from about 15 to about 50% protein, from about 5 to about 40% fat, and from about 5 to about 20% moisture, and an ash content of from about 5 to about 10%.

The UFA and NORC are administered on an extended regular basis for some embodiments, and on a daily basis for one embodiment. Daily administration in accordance with the methods herein may be continued on a extended regular basis.

In one embodiment, the compounds or compositions are administered to the animal in conjunction with one or more therapeutic agents useful for, and in an amount effective for, treating, reducing, or preventing damage of one or more components of the nervous system of an animal. The compositions can also be administered in conjunction with one or more physical treatments for treating, reducing, or preventing damage to the nervous system.

In various embodiments, the therapeutic agent useful for treating, reducing, or preventing damage to an animal's nervous system includes one or more of a corticosteroid, erythropoietin, an immunosuppressive drug, mexiletine, an immunoglobulin, a therapeutic antibody, a therapeutic cell, retinoic acid or a derivative thereof, a microRNA, a neurotrophin, an antidepressant drug, an antiepileptic drug, sodium valproate or a pharmaceutically acceptable salt of valproic acid, a cannabinoid, an anticonvulsant, and lidocaine.

Skilled artisans will appreciate that some of the foregoing represent classes of therapeutic agents and any member of the class may be suitable for use herewith. In presently preferred embodiments, the corticosteroid is methylprednisolone; the immunosuppressive drug is for example, one or more of prednisone, cyclosporine, or azathioprine; the neurotrophin is NTF-3; the antidepressant drug is amitriptyline or other tricyclic antidepressant; the antiepileptic drug is gabapentin, phenytoin, or carbamazepine; and/or the anticonvulsant is pregabalin. Where a therapeutic cell is employed, it is preferably a stem cell, or a cell capable of producing a cell product that is useful for the damaged nervous system component.

Where the methods involve administering the composition to the animal in conjunction with one or more physical treatments, in various embodiments the treatment comprises one or more of exercise, plasmapheresis, orthopedic supports or braces, and/or TENS (Transcutaneous Electrical Nerve Stimulation).

In one embodiment, the composition administered is a pharmaceutical or nutraceutical composition that optionally comprises one or more therapeutic agents useful for treating, reducing, or preventing the damage.

In another embodiment, the UFA and NORC are administered to the animal in conjunction with one or more physical treatments useful for treating, reducing, or preventing the damage of one or more of components of the peripheral nervous system, as described above.

The physical treatments are preferably one or more of exercise, plasmapheresis, orthopedic supports or braces, and/or Transcutaneous Electrical Nerve Stimulation (TENS).

In one embodiment, the treatment may be administered further in conjunction with one or more therapeutic agents also useful for treating, reducing, or preventing the damage, because such combination treatments may be more effective than monotherapies. The therapeutic agent is preferably one or more of a corticosteroid, erythropoietin, an immunosuppressive drug, mexiletine, an immunoglobulin, a therapeutic antibody, a therapeutic cell, retinoic acid or a derivative thereof, a microRNA, a neurotrophin, an antidepressant drug, an antiepileptic drug, sodium valproate or a pharmaceutically acceptable salt of valproic acid, a cannabinoid, an anticonvulsant, and lidocaine.

For such methods to be most useful, the damage is caused by injury, disease, or a systemic or metabolic condition. In one embodiment the animal is an aged animal, preferably a healthy aging animal, and may also be a companion animal. The animal in one embodiment has a phenotype associated with age-related damage of the nervous system. In various embodiments of this and other aspects of the invention, the phenotype includes one or more of an altered sensory function or an altered motor functions, or other altered nervous system function such processing of sensory or motor information.

Altered sensory functions include one or more of an altered mechanoreceptors, chemoreceptors, thermoreceptors, electroreceptors, tactile receptors, or pain receptors, preferably as compared to a control animal not having the phenotype. Altered motor function includes increased reflex time, decreased fine motor control, decreased stability, decreased range of movement, decreased extent of movement, an actual or perceived muscle weakness, paralysis, or muscle wasting, preferably as compared to a control animal not having the phenotype. The phenotype can also be any altered production, storage, release, transmission, reception, propagation, relay, or timing of neurological signals as compared to a control animal not having the phenotype; each of the foregoing preferably as compared to a control animal not having the phenotype.

In a further aspect, the invention provides methods for promoting the health and wellness of animals. The methods comprise identifying an animal susceptible to or suffering from damage to at least one component of the nervous system and administering in conjunction to the animal one or more UFA and one or more NORC in an amount effective for treating, reducing, or preventing damage to at least one component of the nervous system of the animal; thereby promoting the health and wellness of the animal. The preferred UFA and NORC are also described herein, e.g., ALA, EPA, DPA, DHA, and arginine.

In certain embodiments, the damage is caused by physical injury or trauma, disease, or a systemic or metabolic condition. In various embodiments, the animal is a human or companion animal, preferably an adult animal, and more preferably an aging animal.

In various embodiments, the methods further comprise administering one or more B vitamins, one or more antioxidants, or combination thereof to the animal in an amount effective for treating, reducing, or preventing damage to at least one component of the nervous system of an animal. Preferred amounts are described herein.

In another aspect, the invention provides methods for extending the prime years of life for animals. The methods comprise identifying an animal susceptible to or suffering from damage to at least one component of the nervous system and administering to an animal in conjunction one or more UFA and one or more NORC in an amount effective for treating, reducing, or preventing damage to at least one component of the nervous system of the animal; thereby extending the prime for the animal. The preferred UFA and NORC are also described herein, e.g., ALA, EPA, DPA, DHA, and arginine.

In certain embodiments, the damage is caused by physical injury or trauma, disease, or a systemic or metabolic condition. In various embodiments, the animal is a human or companion animal, preferably an adult animal, and more preferably an aging animal.

In various embodiments, the methods further comprise administering one or more B vitamins, one or more antioxidants, or combination thereof to the animal in an amount effective for treating, reducing, or preventing damage to at least one component of the nervous system of an animal. Preferred amounts are described herein.

In a further aspect, the invention provides methods for improving the quality of life of animals. The methods comprise identifying an animal susceptible to or suffering from damage to at least one component of the nervous system and administering to an animal in conjunction one or more UFA and one or more NORC an amount effective for treating, reducing, or preventing damage to at least one component of the nervous system of an animal, thereby improving the quality of life of the animal. The preferred UFA and NORC are described herein, e.g., ALA, EPA, DP A, DHA, and arginine.

In certain embodiments, the damage is caused by physical injury or trauma, disease, or a systemic or metabolic condition. In various embodiments, the animal is a human or companion animal, preferably an adult animal, and more preferably an aging animal.

In various embodiments, the methods further comprise administering one or more B vitamins, one or more antioxidants, or combination thereof to the animal in an amount effective for treating, reducing, or preventing damage to at least one component of the nervous system of an animal. Preferred amounts are described herein.

In another aspect, the invention provides a package useful for containing one or more UFA, NORC, B vitamins, antioxidants, or other components of the invention and an indication that the package contains compounds that are effective for treating, reducing, or preventing damage to the nervous system of an animal. The package comprises at least one material suitable for containing the components and a label affixed to the material containing a word or words, picture, design, acronym, slogan, phrase, or other device, or combination thereof, that indicates that the package contains the components and indicates their use. Typically, such device comprises the words “prevents damage to the nervous system” or “contains ingredients that treat, reduce, or prevent damage to the nervous system” or an equivalent expression printed on the material. Any package configuration and packaging material suitable for containing the components are useful in the invention, e.g., a bag, box, bottle, can, pouch, and the like manufactured from paper, plastic, foil, metal, and the like. In preferred embodiments, the package further comprises UFA, NORC, B vitamins, antioxidants, or other components of the invention. In various embodiments, the package further comprises at least one window that permit the package contents to be viewed without opening the package. In some embodiments, the window is a transparent portion of the packaging material. In others, the window is a missing portion of the packaging material.

The compounds and compositions useful in the methods, including the pharmaceutical compositions and medicaments, are administered to the animal for a time required to accomplish one or more objectives of the invention, e.g., treating, reducing, or preventing damage of one or more components of the peripheral nervous system, including for example a glial cell, a ganglion, myelin, a neuron, or another component of the nervous system. In one embodiment, damage to at least a portion of a neuron that is soma, axon, dendrite, synapse, terminus, receptor, or effector, or damage to a motor plate can be treated, reduced, or prevented. In another embodiment, the glial cell is a Schwann cell, satellite cell, enteric cell, or other glial cell of the peripheral nervous system.

With respect to this or any aspect of the invention, the methods and compositions can also be used for a time required to altering at least one phenotype exhibited by an animal experiencing damage to a component of the nervous system. The compositions are suitable for long-term administration or administration on any schedule compatible with the composition and objective.

### EXAMPLES

The invention can be further illustrated by the following examples, although it will be understood that the examples are included merely for purposes of illustration and are not intended to limit the scope of the invention unless otherwise specifically indicated.

### Example 1

Test Compositions and Animal Groups: Young adult (2-3 months old) male Charles Rivers rats were fed one of four compositions and subjected to one of four treatments as follows: Group A: Control composition. Feeding 1 month+sham surgery+control feeding 1 month (sham control); Group B: Control composition. Feeding 1 month+MCAO+control feeding 1 month (stroke control); Group C: Test composition. Feeding 1 month+MCAO+test diet feeding 1 month (pre+post feeding); and Group D: Control composition. Feeding 1 month+MCAO+test diet feeding 1 month (post feeding only). The Control was a standard rat diet containing 140 g/kg casein, 100 g/kg sucrose, 50 g/kg fiber, 155 g/kg dextrin, 466 g/kg corn starch, 35 g/kg standard salt mix, 40 g/kg soybean oil, 10 g/kg standard vitamin mix, 1.8 g/kg L-cystine and 2.5 g/kg choline chloride. The test composition was the Control Diet supplemented with 2% Arginine, and 2% Menhaden fish oil, B vitamins at 4 times of RDA requirements for rats, and antioxidants (Vitamin E: 500 mg/kg diet, Vitamin C: 150 mg/kg diet, astaxanthin: 100 mg/kg, selenium: 0.40 mg/kg).

The rats were maintained in our animal facility in a temperature-controlled room (22-25°0 C.) with 12-hour dark-light cycles. All rats had free access to laboratory chow and tap water during the acclamation period and to the control or test diet during the study period.

At the end of the 4 weeks of feeding, animals were subjected to a transient (1 hour) middle cerebral artery occlusion followed by reperfusion for 24 hours to induce brain damage by ischemia and hypoxia. For middle cerebral artery (MCA) occlusion and reperfusion, an intraluminal filament model was used. Briefly, the animals were anesthetized with ketamine (60mg/kg) and xylazine (10 mg/kg), then the internal carotid artery (ICA) was exposed, and a 3-0 monofilament nylon suture was introduced into the ICA lumen through a puncture and gently advanced to the distal internal carotid artery (ICA) until proper resistance was felt. After 1 hr, the suture was withdrawn from the ICA and the distal ICA was immediately cauterized.

The rats had one month to recover from the brain damage. One month after MCAO or sham surgery, all the animals were assessed for both cognitive (Morris water maze) and locomotor function (Rotarod).

Morris water maze Test: The apparatus consists of an aluminum tank (130 cm diameter×75 cm deep) filled with white-colored tap water to a depth of 50 cm and maintained at 24° C. The rats were required to locate a platform (10×10 cm) situated 1 cm beneath the surface. A camera (Burle, Lancaster, Pa.) and computerized tracking system (San Diego instruments) were used to record the position of the rats. During the pre-training phase, a black curtain was placed over the tank, and the rats were allowed to swim and climb onto the platform without extra maze cues available. On each trial, the rats were allowed to swim from one end of a straight alley (100×15×60 cm, placed in the middle of the tank) and climb to the hidden platform at the other end of the alley. The rats were left on the platform for 10 s and then were placed in a holding cage for a 5-minute intertrial interval (ITI). This phase included 4 sessions of 5 trials each over a period of 2 days. The morning and afternoon sessions were separated by at least 2 hours. The time (seconds) for the rats to reach the platform was recorded on each trial.

During the acquisition phase, the curtain and alley were removed and the rat was required to locate the platform using spatial cues in a period of 90 s. After a period of 10 s resting on the platform, the rat was placed back in the holding cage for an ITI of 10 min. The acquisition phase had 4 sessions with 5 trials each session, over a period of 2 days. Sessions 5 were carried out after a delay of 24 hours and session 6 started after a 12-hour delay, with the platform still in the original location. The objective was to assess how well the rat can remember the platform location over a delay period. Performance was measured by Latency to platform.

Rotarod Test: Rotarad test was used to evaluate the balance and coordination ability of the rat after recovery from the brain damage. The apparatus is a motor-driven treadmill (Accuscan Instruments) with a nylon cylinder (45 cm length, diameter 3.2 cm) mounted horizontally at a height of 35.5 cm above a padded surface. The cylinder has four, 11-cm wide compartments separated by black acrylic dividers. On a given trial, the rat was placed on the cylinder and a microprocessor-controlled motor rotated the cylinder with an acceleration of 0.5 rpm/sec. The trial ended when the rat fell from the cylinder or a speed of 75 rpm was reached. The latency to fall will be recorded on each of four trials that are spaced at 10-min intervals within each session. The rats received 2 sessions daily until a criterion of stability was reached (i.e., until the average latency to fall did not increase by more than 15% over the last three sessions). The average latency to fall on the last training session was the measure of performance.

The results are shown in Table 1 and Table 2. Referring to the Tables, the data show that the

compositions are effective for preventing and treating nervous system injury.

TABLE 1
Effect of Nutrient Blend on Morris Water Maze Performance after
Recovery from Brain Damage: Latency to platform (seconds)

| Session | Group A | Group B | Group C | Group D |
|---|---|---|---|---|
| 1 | 30.98 | 50.64 | 46.08 | 46.37 |
| 2 | 16.41 | 23.85 | 19.56 | 16.72 |
| 3 | 13.13 | 24.48 | 18.73 | 16.44 |
| 4 | 10.08 | 15.78 | 11.89 | 10.97 |
| 5 | 8.832 | 17.56 | 9.33 | 9.60 |
| 6 | 9.852 | 11.85 | 8.61 | 9.64 |

TABLE 2
Effect of Nutrient Blend on Motor functions (balance and coordination)
after Recovery from Brain Damage: Latency to Fall (seconds)

| Session | Group A | Group B | Group C | Group D |
|---|---|---|---|---|
| 1 | 17.34 | 12.7 | 16.42 | 15.81 |
| 2 | 29.2 | 21.17 | 26.16 | 23.41 |
| 3 | 33.29 | 23.08 | 27.41 | 29.56 |
| 4 | 40.24 | 26.72 | 32.77 | 28.35 |
| 5 | 40.39 | 26.55 | 33.57 | 28.53 |
| 6 | 37.86 | 27.3 | 34.24 | 28.54 |
| 7 | 36.11 | 26.15 | 30.46 | 27.63 |
| 8 | 35.71 | 26.18 | 30.9 | 27.13 |

### Example 2

Rat Experiments: The experimental design is similar to that in Example 1. Five groups of rats are to be used and will be fed different diets as in Example 1. The rats to be used will have peripheral nerve damage exclusive of central nerve system damage. Such damage can be induced in various experimental models such as by tightly ligating spinal nerves, or intoxication of various types, such as using Buckthorn fruit (Karwinskia humboldtiana) or other neurotoxins. Rats will be fed their respective daily diet containing UFA, NORC, and optionally B vitamins and/or antioxidants for a prolonged period (“extended regular basis”).

Various measures of motor functions or nerve function (e.g., nerve conductance velocity) responses for rats in the groups receiving the UFA/NORC diets are measured and compared to the control group not receiving the UFA and NORC diet. The results are expected to show substantially better performance among the rats receiving the protective diets compared to those of the control group.

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims, the invention may be practiced otherwise than as specifically described.

1. A method for treating, reducing, or preventing damage to the nervous system of an animal comprising identifying an animal susceptible to or suffering from damage to at least one component of the nervous system and administering in conjunction to the animal one or more unsaturated fatty acids (UFA) and one or more nitric oxide releasing compounds (NORC) in an amount effective for treating, reducing, or preventing damage to the nervous system of the animal.

2. The method of claim 1 wherein damage to the nervous system comprises damage to a glial cell, a ganglion, myelin, a neuron, or another component of the nervous system.

3. The method of claim 2 wherein the damage is to at least a portion of a neuron that is soma, axon, dendrite, synapse, terminus, receptor, or effector, or a motor plate.

4. The method of claim 2 wherein the damage is to a glial cell that is a microglia, astrocyte, oligodendrocyte, ependymal cell, radial glia, Schwann cell, satellite cell, enteric glial cel, or other glial cell.

5. (canceled)

6. The method of claim 2 wherein the damage is to a component of the animal's brain.

7. The method of claim 2 wherein the damage is to a component of the animal's spinal cord.

8. The method of claim 2 wherem the damage is to a component of the animal's peripheral nervous system.

9. The method of claim 1 wherein the damage is caused by trauma to the brain or spinal cord.

10. The method of claim 1 wherein the damage is caused by physical injury, disease, systemic status of the animal, metabolic status of the animal, or combination thereof.

11. (canceled)

12. The method of claim 1 wherein the damage is associated with normal aging.

13. The method of claim 1 wherein the damage is associated with trauma.

14. (canceled)

15. (canceled)

16. (canceled)

17. (canceled)

18. (canceled)

19. The method of claim 1 wherein the animal is a human or companion animal.

20. The method of claim 19 wherein the companion animal is a canine or a feline.

21. (canceled)

22. The method of claim 1 wherein the UFA are ALA, EPA, DPA, DHA, or another n-3 fatly acid from any source.

23. The method of claim 1 wherein the UFA are from a fish oil source.

24. The method of claim 1 wherein the NORC are arginine, a nitric oxide-releasing analog or derivative of arginine, citulline, or ornithine.

25. The method of claim 1 wherein the UFA and the NORC are administered to the animal in amounts of from about 0.001 g/kg to about 50 g/kg.

26. (canceled)

27. The method of claim 1 further comprising administering in conjunction to the animal one or more B vitamins in an amount effective for treating, reducing, or preventing the damage.

28. (canceled)

29. The method of claim 1 further comprising administering in conjunction to the animal one or more antioxidants in an amount effective for treating, reducing, or preventing the damage.

30. (canceled)

31. (canceled)

32. The method of claim 1 further comprising administering in conjunction to the animal one or more B vitamins and one or more antioxidants in an amount effective for treating, reducing, or preventing the damage.

33. The method of claim 32 wherein the B vitamins are administered in an amount of from about 0.1 to about 40 times the recommended daily requirement of B vitamins and the antioxidants are administered in an amount of from about 0.1 to about 10 times the recommended daily allowance for the antioxidants.

34. The method of claim 1 wherein the UFA and NORC are administered as part of a composition formulated as a human food composition, a pet food composition, or a dietary supplement.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. The method of claim 1 wherein die UFA and NORC are administered in a pharmaceutical or nutraceutieal composition that further comprises one or more antioxidants ami one or more B vitamins.

52. The method of claim 51 wherein the composition comprises from about 0.0001 to about 25% of antioxidants and from about 0.1 to about 40 times the recommended daily requirement of B vitamins.

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)
